(19) United States
(12) Patent Application Publication
(10) Pub. No.: US 2004/0171862 A1
(43) Pub. Date: Sep. 2, 2004

(54) Method for producing optically active cyanohydrins and their corresponding acids

(76) Inventors: Bettin Kirschbaum, Frankfurt (DE); Rainer Fell, Gersthofen (DE)

Correspondence Address: CLARIANT CORPORATION, INTELLECTUAL PROPERTY DEPARTMENT, 4000 MONROE ROAD, CHARLOTTE, NC, 28205, US

(21) Appl. No.: 10/482,246

(22) PCT Filed: Jun. 18, 2002
(86) PCT No.: PCT/EP02/06681

(30) Foreign Application Priority Data
Jun. 30, 2001 (DE) 101 31 810.3

(51) Int. Cl. C07C 253/06

(52) U.S. Cl. 558/341

(57) ABSTRACT

The invention relates to a method for producing optically active cyanohydrins and the corresponding α-hydroxy-carboxylic acids, starting from an aldehyde, hydrogen cyanide and an optically active vanadyl-salen catalyst, whereby the reaction mixture is reacted at a temperature of between 0 and 60° C. Between 0.8 and 10 equivalents of hydrogen cyanide and between 0.0001 and 0.05 equivalents of vanadyl-salen catalyst in relation to the aldehyde, (concentration of between 0.5 and 4 mol/litre solvent), are preferably used. After said reaction the optically active cyanohydrin or after an acid hydrolysis the corresponding optically active α-hydroxycarboxylic acid can be isolated with a surplus of enantiomers. The vanadium catalyst used in the invention contains a salen ligand, whereby the ratio salen ligand: vanadium (IV) in the catalyst ranges between 1.4:1 and 10:1.

The invention relates to a process for preparing optically active cyano-hydrins by means of an optically active vanadyl catalyst. Optically active cyanohydrins and their subsequent products, for example optically active α-hydroxy carboxylic acids, serve as building blocks for obtaining biologically active substances which find use, for example, in the pharmaceutical or agrochemical industry. Cyanohydrins are obtainable by various chemical reactions, as described in Top. Curr. Chem. 1999, 200, 193-226.

One means of synthesizing optically active cyanohydrins is to convert aldehydes in the presence of molecules having “CN” groups (HCN, MCN where M is alkali metal, trimethylsilyl cyanide—also referred to as TMSCN, cyanohydrins, e.g. acetone cyanohydrin) and an optically active catalyst to (S)— or (R)-cyanohydrins, (Compr. Asymmetric Catal. I-III, 1999 (2), Ch. 28).

A series of catalysts allows the enantioselective addition of the CN group to aldehydes, but primarily using trimethylsilyl cyanide as the CN source (I. P. Holmes, H. B. Kagan, Tetrahedron Left. 2000, 41, 7457-7460. Y. Hamashima et al., Tetrahedron 2001, 57, 805-814. E. Leclerc et al., Tetrahedron: Asymmetry, 2000,11, 3471-3474.).

For instance, when optically active transition metal catalysts, for example titanium-salen complexes, are used, an enantioselective addition of trimethylsilyl cyanide to aldehydes is known (Y. Belokon, J. Chem. Soc., Perkin Trans. 1 1997, 1293-1295. Y. N. Belokon et al. J. Am. Chem. Soc. 1999, 121, 3968-3973.).

Y. N. Belokon et al. (J. Am. Chem. Soc. 1999, 121, 3970) reported that there is no reaction with titanium-salen complexes when free HCN is used under the same conditions (at −80° C.). Y. N. Belokon et al. additionally report in Eur. J. Org. Chem. 2000, 2655-2661 that titanium-salen complexes, when free HCN is used, result even at room temperature in only a very slow reaction in comparison to the use of TMSCN. Good conversions and enantioselectivities consequently typically require low temperatures (−80° C.) and TMSCN as the cyanide source.

Although vanadyl-salen complexes catalyze the reaction of aldehydes with trimethylsilyl cyanide in principle with higher enantioselectivity than the corresponding titanium-salen catalysts (Y. N. Belokon, M. North, T. Parsons, Org. Lett. 2000, 2, 1617-1619), only the use of TMSCN as the cyanide source is known here.

A CN source such as trimethylsilyl cyanide is little suited to industrial use, since it is expensive and additionally causes large amounts of silicon wastes. The realization of low temperatures such as −80° C. in industrial application is likewise expensive and not very practical.

It is therefore an object of the present invention to provide a process which overcomes the above-outlined difficulties and limitations with regard to the CN source to be used and reaction temperature, and which can additionally be realized industrially in a simple manner, without entailing great cost and inconvenience.

The present invention achieves this object and relates to a process for preparing optically active cyanohydrins by reacting aldehydes with a CN source in an organic solvent in the presence of an optically active vanadyl catalyst at a temperature in the range from 0 to 60° C., said catalyst containing a salen ligand and the catalyst containing from 1.4 to 10 equivalents of a salen ligand, based on one equivalent of vanadium(IV).

In a preferred embodiment, the invention relates to the preparation of cyanohydrins of the formula (II)

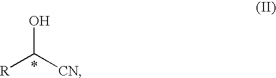

where the optically active center * has the absolute configuration (R) or (S), R is an optionally branched alkyl, alkenyl or alkynyl radical of chain length C₁ to C₂₀, in particular C₁ to C₈, or a radical of the formula (IIa)

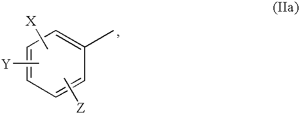

where X, Y and Z are each independently the same or different and are H, F, Cl, Br, I, OH, NH₂, O(C₁-C₄-alkyl), OCOCH₃, NHCOCH₃, NO₂ or C₁-C₄-alkyl.

Cyanohydrins of the formula (II) are obtained by reacting an aldehyde of the formula (I)

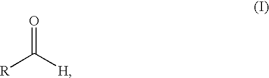

where R is as defined above,

in accordance with the above-specified provisions.

In a further preferred embodiment, aldehydes of the formula (Ia)

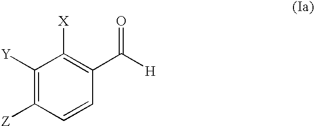

are used.

In the formula (Ia), X is preferably F, Cl, Br, I, OH O(C₁-C₄-alkyl), OCOCH₃, NHCOCH₃, NO₂ or C₁-C₄-alkyl and Y and Z are each H, or X and Y are each H and Z is OH, or X is H and Y and Z are each OH.

The CN source used may be pure hydrocyanic acid, acid-stabilized hydro-cyanic acid or a cyanohydrin, in particular acetone cyanohydrin. The cyanohydrin present in the reaction mixture may optionally be converted by hydrolysis directly to the corresponding α-hydroxy carboxylic acid.

One advantage of the process according to the invention is that it is possible to not only use the aldehydes in comparatively less concentrated amounts than hitherto customary, for example 0.1 mol of aldehyde/liter, but also to carry out the reaction with considerably higher aldehyde concentrations, for example from 2.0 mol of aldehyde/liter up to 10 mol of aldehyde/liter, preferably from 2 to 4 mol of aldehyde/liter. Accordingly, the space-time yield for stereoselective cyanohydrin reactions is unusually high.

According to the invention, the reaction with HCN is carried out in an organic solvent. Suitable for this purpose are in principle all organic solvents or solvent mixtures which behave inertly under the conditions of the reaction.

Particularly suitable as solvents are C₆-C₁₀ aromatic and C₁-C₁₀ aliphatic, optionally halogenated hydrocarbons or solvent mixtures thereof, and aliphatic ethers having from 1 to 5 carbon atoms per alkyl radical, or cyclic ethers having from 4 to 5 carbon atoms in the ring.

Especially suitable are aromatic, optionally substituted C₆-C₁₀, preferably C₆-C₉, hydrocarbons, for example toluene, ortho-, meta- and/or para-xylene, chlorinated aliphatic or aromatic hydrocarbons such as methylene chloride, dichloroethane, trichloroethane, chloroform, chlorobenzene, dichlorobenzene and trichlorobenzene, or ethers, for example diethyl ether, di-n-propyl ether, diisopropyl ether, di-n-butyl ether and methyl tert-butyl ether.

For each mole of aldehyde of the formula (I), from 0.8 to 10.0, in particular from 1.0 to 5.0, preferably from 1.5 to 3.5, more preferably from 2.0 to 3.0, mol of HCN are used. However, it is also possible to carry out the reaction at from 0.5 to 20 mol of HCN/mol of aldehyde.

The process according to the invention is carried out at a temperature of from 0 to 60° C., preferably from 10 to 50° C., in particular from 20 to 40° C.

Suitable catalysts are vanadyl-salen complexes consisting of salen ligands of the general formula (III) and vanadium in the oxidation state (IV).

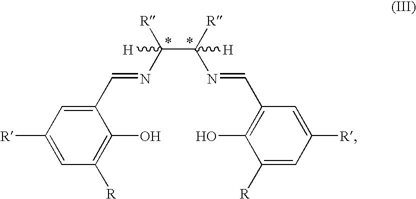

*R,R— or S,S-enantiomer

The R, R′ and R″ radicals of the salen ligands of the general formula (III) may each independently be hydrogen, branched or unbranched C₁-C₁₀ alkyl radicals, in particular a methyl or tert-butyl radical, or an O(C₁-C₄-alkyl) group, in particular a methoxy group, or halogens, in particular Cl, a substituted aryl group, in particular a phenyl group, or —(CH₂)ₘ—, where m may be an integer between 1 and 8.

The salen ligand: vanadium (IV) ratio in the catalyst is in the range from 1.4:1 up to 10:1, preferably in the range from 1.4:1 to 5:1, in particular in the range from 1.4:1 to 3:1.

Such catalysts are described in the German Patent Application P . . . (Internal Number R. 4485) which has the same priority date and had not been published in advance of the present application, and is explicitly incorporated herein by way of reference.

The catalyst is prepared by reacting vanadyl sulfate with from 1.4 to 10, in particular from 1.4 to 5, equivalents of the appropriate salen ligand.

The catalysts contain salen ligands of the formula (III) and vanadium in the oxidation state (IV) and are preferably synthesized in alcohols in a hetero-geneous reaction environment or in a chlorohydrocarbon/alcohol mixture in a heterogeneous reaction environment.

To carry out the process according to the invention, the vanadyl-salen catalyst is mixed with the aldehyde and HCN in a suitable solvent. From 0.00005 to 0.05 equivalent of catalyst, preferably from 0.0001 to 0.01 equivalent of catalyst, based on the aldehyde is used.

As mentioned at the outset, the reaction is carried out at from 0 to 60° C., in particular at from 10 to 50° C., preferably at from 20 to 40° C. In many cases, it has proven advantageous to allow the reaction to proceed at room temperature.

The aldehyde is added in a concentration of from 0.1 to 10.0, in particular from 0.5 to 5.0, preferably from 1.0 to 4.5, mol of aldehyde/liter of the reaction mixture. In a multitude of cases, it has proven advantageous to carry out the reaction with HCN at an aldehyde concentration of from 2.0 to 4.0 mol/liter.

On completion of the reaction, if desired, the optically active cyanohydrin can be isolated from the reaction mixture and optionally also purified. Using toluene as the solvent, the optically active cyanohydrin can be crystallized out, for example, under cooled conditions, preferably at temperatures in the range from −20° C. to 10° C.

However, the optically active cyanohydrin, optionally in the form of the reaction mixture, can also be converted, for example by acid hydrolysis, to the corresponding optically active a-hydroxy carboxylic acid. For the acidic hydrolysis, it is customary to use strong mineral acids, such as conc. HCl or aqueous sulfuric acid. In the course of the hydrolysis, it is necessary to ensure good mixing of the aqueous phase in which the acid is present and the organic phase in which the optically active cyanohydrin is present. By adding an ether (e.g. diisopropyl ether) or a phase transfer catalyst (e.g. a polyethylene glycol), the rate of the hydrolysis reaction can be increased.

The process according to the invention surprisingly enables aldehydes to be converted with high conversions and good ee values to the optically active cyanohydrins, both of the (S) and of the (R) series. In particular, it is possible to convert substrates which are particularly difficult, for example, for enzymatic processes, such as benzaldehydes substituted in the 2-position, e.g. 2-chlorobenzaldehyde, as desired to the corresponding optically active (S)— or (R )-cyanohydrins with good success by means of the process according to the invention.

### EXAMPLES

The examples which follow describe the invention in detail, without restricting it.

The ee values of the cyanohydrins obtained were determined by gas chromatography using a β-cyclodextrin column after derivatization with acetic anhydride/pyridine.

The VO-salen complex used in the examples which follow is a complex prepared from salen ligands of the formula (III) and vanadium in the oxidation state (IV), for example vanadyl(IV) sulfate.

Preparation of the VO-salen complexes with the salen ligands IIIa-d

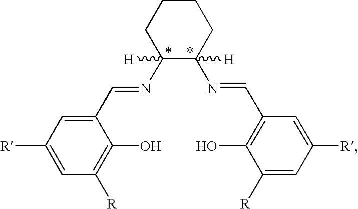

(IIIa), R,R-enantiomer, R═R′=tert-butyl

(IIIb), S,S-enantiomer, R═R′=tert-butyl

(IIIc), R,R-enantiomer, R=tert-butyl, R′=methyl

(IIId), R,R-enantiomer, R=tert-butyl, R′=methoxy

### Example 1

Synthesis of VO-Salen Complex with the Salen Ligand (IIIa):

5.46 g (0.01 mol) of (R,R)-2,2′-[1,2-cyclohexanediyl)bis(nitrilomethylidyne)]-bis[4,6-di-tert-butyl)phenol] are initially charged in 50 ml of ethanol and admixed with 1.14 g (0.0045 mmol) of vanadyl sulfate pentahydrate. After three hours under reflux and complete conversion (TLC monitoring), the solvent is distilled off, the residue taken up in 200 ml of dichloromethane and the solution washed with 100 ml of water. After phase separation, drying of the solution with sodium sulfate and distilling off the solvent, 5.4 g of bright green, amorphous powder (yield: 96% of theory) are obtained.

Characterization:

| | |
|---|---|
| Color | bright green |
| Melting point | 208° C., with decomposition |

[α]_{D}²⁰ = −300 (c = 0.01; CHCl₃)

| | |
|---|---|
| paramagnetic | |
| IR (KBr) | ν = 2950 (s), 2870 (m), 2350 (w), 2320 (w), 1610 (vs), |
| | 1550 (m), 1270 (s) [cm⁻¹]. |

### Comparative Example 1

Synthesis of VO-Salen Complex with the Salen Ligands (IIIa):

Use of the Salen Ligands According to the Prior Art in the Ratio of 1:1

5.56 g (0.01 mol) of (R,R)-2,2′-[1,2-cyclohexanediyl)bis(nitrilomethylidyne)]-bis[4,6-di-tert-butyl)phenol] are initially charged in 50 ml of ethanol and admixed with 2.53 g (0.01 mol) of vanadyl sulfate pentahydrate. After three hours under reflux and complete conversion (TLC monitoring), the solvent is distilled off, the residue taken up in 200 ml of dichloromethane and the solution washed with 100 ml of water. After phase separation, drying of the solution with sodium sulfate and distilling off the solvent, 7.7 g of dark green, amorphous powder (yield: 81% of theory) are obtained.

Characterization (cf. Y. N. Belokon: Tetrahedron 57, 2001, 777):

| | |
|---|---|
| Color | dark green |
| Melting point | 233° C. |

[α]_{D}²⁰ = 1000 (c = 0.01; CHCl₃)

| | |
|---|---|
| diamagnetic | |
| IR (KBr) | ν = 2950 (s), 2870 (m), 2350 (w), 2320 (w), 1610 (vs), |
| | 1550 (m), 1250 (vs), 1210 (s), 1010 (m) [cm⁻¹]. |

### Example 2

Synthesis of VO-Salen Complex with the Salen Ligand (IIIa):

8.0 g (0.015 mol) of (R,R)-2,2′-[1,2-cyclohexanediyl)bis(nitrilomethylidyne)]-bis[4,6-di-tert-butyl)phenol] are initially charged in 200 ml of ethanol and admixed with 2.5 g (0.01 mol) of vanadyl sulfate pentahydrate. After two hours under reflux and complete conversion (TLC monitoring), the solvent is distilled off, the residue taken up in 200 ml of dichloromethane and the solution washed with 100 ml of water. After phase separation, drying of the solution with sodium sulfate and distilling off the solvent, 8.2 g of green, amorphous powder (yield: 87% of theory, based on a complex having vanadium:salen ligand=1:2).

### Examples 3 to 5

Syntheses of the VO-Salen Complexes with the Salen Ligands (IIIb-d)

These catalysts were prepared from the corresponding ligands (IIIb-c) in a similar manner to Example 2.

| | |
|---|---|
| Yield for IIIb: | 82% (Example 3) |
| Yield for IIIc: | 86% (Example 4) |
| Yield for IIId: | 89% (Example 5) |

Reaction of Aldehydes I with VO-Salen Complexes

### Example 6

Conversion of Benzaldehyde Using VO-Salen Complexes from Example 2:

A flask equipped with stirrer and internal thermometer is initially charged with 150 ml of dichloromethane. 0.46 g (0.40×10⁻³ mol) of (R,R)—VO salen complex from Example 2 and 15.9 g (0.15 mol) of benzaldehyde (freshly dist.) are added in succession with stirring. 10.1 g (0.375 mol) of hydrocyanic acid are then added all at once. The dark green, homogeneous solution is stirred at room temperature for 24 hours in the closed apparatus. The conversion is quantitative according to GC.

Hydrolysis:

After distilling off the solvent, 100 g of concentrated hydrochloric acid (36.5%) are added to the mixture and stirred at 50-60° C. for 6 hours.

Subsequently, 100 ml of water is added to the reaction mixture and the mixture is extracted twice with 100 ml of DIPE (diisopropyl ether) each time. The combined organic phases are concentrated to dryness. The crude product is recrystallized from 150 ml of toluene.

The yield is 11.4 g of (S)-mandelic acid (68% of theory based on benzaldehyde; 88% ee).

### Example 7

Conversion of 2-chlorobenzaldehyde Using VO-Salen Complex from Example 1:

A flask equipped with stirrer and internal thermometer is initially charged with 150 ml of toluene. 0.08 g (0.15×10⁻³ mol) of (R,R)—VO salen complex from Example 1 and 21.1 g (0.15 mol) of 2-chlorobenzaldehyde (freshly dist.) are added in succession with stirring. 10.1 g (0.375 mol) of hydrocyanic acid are then added all at once. The dark green, homogeneous solution is stirred at room temperature for 24 hours in the closed apparatus. The conversion according to GC is: 98%; 73% ee for the (S)-2-chloromandelic acid cyanohydrin.

Hydrolysis:

150 ml of diisopropyl ether and 112.5 g of concentrated hydrochloric acid (36.5%) are added to the reaction mixture. The mixture is stirred at 60° C. for 6 hours. Two phases form.

Subsequently, 100 ml of water are added to the reaction mixture and the organic phase is removed. The aqueous phase is extracted twice with 100 ml of DIPE each time. The combined organic phases are concentrated to dryness. The crude product is recrystallized from 150 ml of toluene.

The yield is 15.4 g of (S)-2-chloromandelic acid (55% of theory, based on 2-chlorobenzaldehyde; 96% ee).

### Comparative Example 7

Conversion of 2-chlorobenzaldehyde with VO-Salen Complex from Comparative Example 1 (Org. Lett. 2000, 2,1617-1619):

A flask equipped with stirrer and internal thermometer is initially charged with 150 ml of toluene. 0.09 g (0.15×10⁻³ mol) of (R,R)—VO salen complex from Comparative Example 1 and 21.1 g (0.15 mol) of 2-chlorobenzaldehyde (freshly dist.) are added in succession with stirring. 10.1 g (0.375 mol) of hydrocyanic acid are then added all at once. The dark green, homogeneous solution is stirred at room temperature for 24 hours in the closed apparatus. The conversion according to GC is: 57%; 25% ee for the (S)-2-chloromandelic acid cyanohydrin.

### Example 8

Conversion of 2-chlorobenzaldehyde Using VO-Salen Complex from Example 3:

In a similar manner to Example 6, 21.1 g (0.15 mol) of 2-chlorobenzaldehyde are converted using 0.009 g (0.08×10⁻³ mol) of (S,S)—VO-salen complex from Example 3. The yield is 18.7 g of (R)-2-chloromandelic acid (67% of theory based on 2-chlorobenzaldehyde; 92% ee).

### Examples 9 and 10

Conversion of 2-chlorobenzaldehyde Using VO-Salen Complexes from Examples 4 and 5:

In a similar manner to Examples 6, 21.1 g (0.15 mol) of 2-chlorobenzaldehyde are converted using (S,S)—VO-salen complexes from Examples 4 and 5.

The conversions, yields and ee values can be taken from the table.

| | | Cyanohydrin | | | |
|---|---|---|---|---|---|
| | | Conversion | | Acid | |
| | | to | ee | Yield in [%] | |
| | Complex with | cyanohydrin | (cyanohydrin) | based on | ee (acid) in |
| Example | salen ligands | in [%] | in [%] | aldehyde | [%] |
| 6 | IIIa | Not | Not | 68 | 88 |
| | | determined | determined | | |
| 7 | IIIa | 98 | 73 | 55 | 96 |
| 8 | IIIb | Not | Not | 67 | 92 |
| | | determined | determined | | |
| 9 | IIIc | 91 | 64 | 45 | 99 |
| 10 | IIId | >99 | 75 | 79 | 83 |

### Example 11

Conversion of the 2-chlorobenzaldehyde Using VO-Salen Complex from Example 1:

A flask equipped with stirrer and internal thermometer is initially charged with 150 ml of diisopropyl ether, 0.09 g (0.08×10⁻³ mol) of (R,R)—VO salen complex from Example 1 and 21.1 g (0.15 mol) of 2-chlorobenzaldehyde (freshly dist.) are added in succession with stirring. 10.1 g (0.375 mol) of hydrocyanic acid are then added all at once. The dark green, homogeneous solution is stirred at room temperature for 24 hours in the closed apparatus. The conversion according to GC is: 99%; 70% ee for the (S)-2-chloromandelic acid cyanohydrin.

### Example 12

Conversion of 2-fluorobenzaldehyde Using VO-Salen Complex from Example 1:

A flask equipped with stirrer and internal thermometer is initially charged with 75 ml of toluene. 0.09 g (0.08×10⁻³ mol) of (R,R)—VO salen complex from Example 1 and 12.3 g (0.15 mol) of 2-fluorobenzaldehyde (freshly dist.) are added in succession with stirring. 10.1 g (0.375 mol) of hydrocyanic acid are then added all at once. The dark green, homogeneous solution is stirred at room temperature for 24 hours in the closed apparatus. The conversion according to GC is: 99%; 55% ee for the (S)-2-fluoromandelic acid cyanohydrin.

### Examples 13 to 15

Conversion of Fluorobenzaldehydes with VO-Salen Complex from Example 1:

In a similar manner to Example 12, in each case 0.15 mol of 4-fluoro-benzaldehyde, 2,4-difluorobenzaldehyde and 2.6 difluorobenzaldehyde respectively are converted using the VO-salen complex from Example 1.

### Example 13

4-fluorobenzaldehyde reacts to give (S)-4-fluorobenzaldehyde cyanohydrin in a 97% conversion and 73% ee.

### Example 14

2,4-difluorobenzaldehyde reacts to give (S)-2,4-difluorobenzaldehyde cyanohydrin in a 94% conversion and 79% ee.

### Example 15

2,6-difluorobenzaldehyde reacts to give (S)-2,6-difluorobenzaldehyde cyanohydrin in a 99% conversion and 44% ee.

What is claimed is:

1. A process for preparing optically active cyanohydrins by reacting an aldehyde with a CN source in an organic solvent in the presence of an optically active vanadyl catalyst at a temperature in the range from 0 to 60° C., said catalyst containing a salen ligand and the salen ligand: vanadium (IV) ratio in the catalyst being in the range from 1.4:1 to 10:1.

2. The process as claimed in claim 1, wherein cyanohydrins of the formula (II) 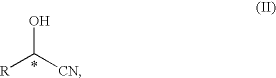
where the optically active center * has the absolute configuration (R) or (S), R is an optionally branched alkyl, alkenyl or alkynyl radical of chain length C₁ to C₂₀ or a radical of the formula (IIa) 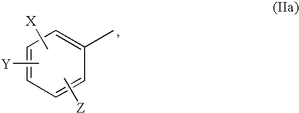
where X, Y and Z are each independently the same or different and are H, F, Cl, Br, I, OH, NH₂, O(C₁-C₄-alkyl), OCOCH₃, NHCOCH₃, NO₂ or C₁-C₄-alkyl,
by reacting an aldehyde of the formula (I) 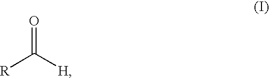
where R is as defined above.

3. The process as claimed in claim 1 and/or 2, wherein an aldehyde of the formula (Ia) 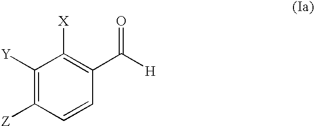
is used.

4. The process as claimed in at least one of the preceding claims, wherein the CN source used is pure hydrocyanic acid, acid-stabilized hydrocyanic acid or a cyanohydrin, in particular acetone cyanohydrin.

5. The process as claimed in one or more of the preceding claims, wherein the organic solvent is a C₁-C₁₀ aliphatic or C₆-C₁₀ aromatic, optionally halogenated hydrocarbon or mixtures thereof, or an open-chain or cyclic aliphatic ether having in each case from 1 to 5 carbon atoms per alkyl radical or from 4 to 5 carbon atoms per ring.

6. The process as claimed in one or more of the preceding claims, wherein the optically active vanadyl catalyst contains salen ligands of the general formula (III) and vanadium in the oxidation state (IV), and R, R′ and R″ are each independently hydrogen, branched or unbranched C₁-C₁₀-alkyl radicals or an O(C₁-C₄-alkyl) group or halogens or an aryl group or —(CH2)ₘ- where m=1 to 8,

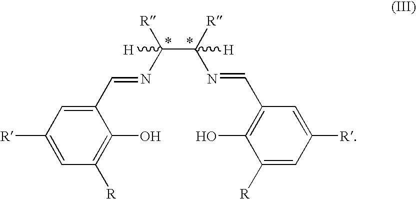

7. The process as claimed in one or more of the preceding claims, wherein from 0.8 to 10 mol of HCN are used per mole of aldehyde.

8. The process as claimed in one or more of the preceding claims, wherein from 0.0001 to 0.05 mol of optically active vanadyl catalyst is used per mole of aldehyde.

9. The process as claimed in one or more of the preceding claims, wherein the aldehyde is used in a concentration of from 0.1 to 10.0 mol of aldehyde/liter of reaction mixture.
